(19) United States
(12) Patent Application Publication
(10) Pub. No.: US 2023/0220154 A1
(43) Pub. Date: Jul. 13, 2023

(54) POLYHYDROXYALKANOATE (PHA) CAKE

(71) Applicant: Meredian, Inc., Bainbridge, GA (US)

(72) Inventors: Joe B. Grubbs, III, Bishop, GA (US); Karson Durie, Athens, GA (US); Michael J. Joyce, Athens, GA (US)

(73) Assignee: Meredian, Inc., Bainbridge, GA (US)

(21) Appl. No.: 18/153,588

(22) Filed: Jan. 12, 2023

Related U.S. Application Data
(60) Provisional application No. 63/266,702, filed on Jan. 12, 2022.

(51) Int. Cl. C08G 63/06; C08G 63/90; C12P 7/625

(52) U.S. Cl. CPC C08G 63/06; C08G 63/90; C12P 7/625

(57) ABSTRACT

A cake material is disclosed, made up of polyhydroxyalkanoate (PHA) cake that is formed directly from biomass and subsequent purification processes absent any heated drying step, with a moisture content of no less than about 5% by weight, and a Dv (90) particle size of no more than about 8 microns.

### FIELD

This invention relates to the field of polyhydroxyalkanoate (PHA) production. More particularly, this invention relates to the production of a novel form of PHA, referred to as PHA cake herein.

### INTRODUCTION

Polyhydroxyalkanoate (PHA) is typically produced by the fermentation of biomass of bacteria or other microorganisms in a bioreactor. First, PHA is synthesized by and accumulated in the cells of the microorganisms. Then, after the PHA is grown in biomass, there is a multi-step process to lyse the bacteria and separate the PHA from the cell debris. Finally, the biomass is dried, such as in an oven, and processed into a dry powder.

The dry powder can be stored and shipped but must be re-wet before use in suspension, emulsion, dispersion, or colloidal forms. Re-wetting typically requires sonication, ultrasonication, low or high shear, and other processes to break the dried particles down into a smaller particle size, and to enable surfactants to wet-out particles. These processes used to re-wet the material take substantial time and energy, and can result in significant foaming. Like all powders, dried PHA includes hazards such as explosion and inhalation. Further, later hydration or other solvation of the dried powder may increase the level of additives such as dispersing and wetting agents (surfactants) to get the particles to disperse and wet-out. These levels of additives tend to decrease the regulatory acceptance, biodegradability, and compostability of the material. The life-cycle assessment of PHA formed into powder is diminished due to the required increased energy and water consumption resulting from the use of drying ovens to create PHA powder, and additional water for re-wetting processes. This overall, reduces the sustainability of PHA powder materials.

Therefore, what is needed is a method of producing PHA that tends to reduce issues such as those introduced above, at least in part.

### DESCRIPTION

The above and other needs are met by a method of forming polyhydroxyalkanoate (PHA) that eliminates heated drying processes, need for re-wetting, increased use of surfactants for dispersing, increased energy usage for high shear dispersing/sonication, significant particle agglomeration resulting in increased particle size as compared to that of those resulting from post lysis. The resulting PHA is newly referred to as PHA cake. The term “cake” is new in this disclosure and has not heretofore been used in the PHA processing industry to describe an end-use material having the properties described above.

PHA cake made according to the methods described here can be either water or solvent-based and is well-suited for formulation of dispersions, colloids, suspensions, coatings, and similar materials. Further, this material is well suited to be used as is into processes which utilize organic and inorganic solvent systems. Examples of formulations made using this cake material include those such as barrier coatings and surface coatings. Further examples for the use of this material include the inclusion or embedding of the electrolytes or material into or onto paper products, dispersions, colloids, emulsions, films, and heat seals. The PHA product is a dispersion of PHA in a liquid, such as water or ethanol, but acts much like an emulsion.

The PHA is created using steps similar to a normal biomass process, but after lysis and protein extraction, the PHA is washed back and forth in an alternating manner with organic and inorganic solvents, such as an alcohol and water, with pressing, terminal filtration, cross flow filtration, decanting, or combination of the two between the washing steps. Blowing air or nitrogen preferably at room temperature can also be performed to yield a PHA cake with no less than about 5% liquid by weight, and in some embodiments, about 60 (+/−10) % PHA and 40 (+/−10) % liquid, by weight.

The final PHA cake product can then be used in a variety of different ways, such as being placed in a mixer to which water, surfactants, preservatives, rheology modifiers, thickening agents, wetting agents, defoamers, biocides, fillers, binders, and dispersing agents are added. These products can then be used in a variety of different ways, as described elsewhere herein.

By not undergoing a heated drying process with elevated temperatures, the PHA cake retains a smaller PHA particle size, when compared with a standard PHA product that has been baked dry to a solid. The smaller particle size further enables better wetting of the PHA particles, reduction in the time/energy for dispersing of particles, reduction in the use of additives (i.e., surfactants) to prevent flocculation and particle agglomerate, and ultimately increases processability, efficiency, and the sustainability of the PHA material and resulting materials made during the application of the PHA cake in commercial processes. Small particles will also increase the film formation propensity of PHA particles when they are brought to the melting temperature and begin to flow onto the substrate to which they have been applied.

PHA cake is better suited for developing aqueous PHA (dispersion, emulsion, colloid, suspension, coating) formulations. Keeping the PHA in a wetted state reduces particle agglomeration, and flocculation, resulting in smaller particles that wet more easily, disperse, and otherwise form aqueous formulations. This approach retains the particle size in the PHA cake to less than about fifteen microns in diameter, with about 90% of the particles falling below 8 microns in size. Further, keeping the PHA in cake form without heated drying reduces the cost of manufacture. PHA materials formed using cake will result in minimum film formation temperature reduction as, reduced dewatering rates, increased rheological profiles, and increased barrier performance as compared to those made using PHA having been dried to a powder state.

The PHA cake can be used in a commercial setting to develop aqueous (or any solvent-based) coating materials. Coating describes any PHA-containing dispersion, solution, emulsion, colloid, or suspension. The PHA cake itself can be sold to a customer for future formulation or formulated after manufacture and before sale to a customer. Some end-users of PHA cake include chemical manufacturing companies, paper and substrate manufacturing companies, molded fiber packaging companies, converting companies, and brand owners.

As used herein, the phrase “at least one of A, B, and C” means all possible combinations of none or multiple instances of each of A, B, and C, but at least one A, or one B, or one C. For example, and without limitation: Ax1, Ax2+Bx1, Cx2, Ax1+Bx1+Cx1, Ax7+Bx12+Cx113. It does not mean Ax0+Bx0+Cx0.

The foregoing description of embodiments for this invention has been presented for purposes of illustration and description. It is not intended to be exhaustive or to limit the invention to the precise form disclosed. Obvious modifications or variations are possible in light of the above teachings. The embodiments are chosen and described in an effort to provide illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. Polyhydroxyalkanoate (PHA) cake that is formed directly from biomass and subsequent purification processes absent any heated drying step, with a moisture content of no less than about 5% by weight, and a Dv (90) particle size of no more than about 8 microns.

2. PHA cake that is formed directly from biomass and subsequent purification processes absent any heated drying step, with a moisture content of no less than about 5% by weight, and a Dv (90) particle size greater than about 8 microns.

3. The PHA cake of claim 1, comprising homopolymer, copolymer, block copolymer, branched copolymer, and terpolymer PHAs or combinations thereof.

4. The PHA cake of claim 1, comprising at least one of short-chain, medium-chain, and long-chain PHAs including at least one of such as butyrate, propionate, valerate, hexanoate, octanoate, and decanoate.

5. The PHA cake of claim 1, PHA cake comprising a final dry mass content of from about 30% to about 95% by weight.

6. The PHA cake of claim 1, wherein the PHA is formed in a biological process.

7. The PHA cake of claim 1, wherein the PHA is formed by fermentation.

8. The PHA cake of claim 1, wherein the PHA is formed via reactive synthesis.

9. The PHA cake of claim 1, comprising PHA content of from about 30% to about 95% by weight.

10. The PHA cake of claim 1, wherein the PHA is produced from a combination of biological and non-biological processes.

11. The PHA cake of claim 1, wherein the PHA cake is separated and purified from production precursors and then filtered through at least one of mechanical dewatering and mechanical desolventing systems.

12. The PHA cake of claim 1, wherein the PHA cake is mechanically dewatered using at least one of organic solvents, inorganic solvents, and solvent-free systems.

13. The PHA cake of claim 1, wherein the PHA cake is mechanically dewatered using a multi-step process.

14. The PHA cake of claim 1, wherein the PHA cake is used in a non-extrusion-based process or application.

15. The PHA cake of claim 1, wherein the PHA cake is used in at least one of an aqueous coating, a solvent coating, emulsion, dispersion, colloid, electrolyte, and suspension.

16. The PHA cake of claim 1, wherein the PHA cake is used in at least one of an aqueous coating, a solvent coating, a dispersion, a colloid, a suspension, and an emulsion, which is used for at least one of inks, barrier coatings, surface coatings, embedded coatings, paper products, films, heat seals, cosmetics, personal care, home care, water treatment, filtration, media, water resistant coatings, and oil resistant coatings.

17. Polyhydroxyalkanoate (PHA) cake formed by:
biological production of PHA,
lysis,
cellular debris removal,
purification of the PHA with alternating washings using organic and inorganic liquids,
pressing/decanting of the PHA between the washings, and
partial drying of the PHA with room temperature gas and without any heated drying step,
thereby producing the PHA cake with a moisture content of no less than about 5% by weight, and a tuned application-specific particle size.
